(19) United States
(12) Patent Application Publication
(10) Pub. No.: US 2025/0318948 A1
(43) Pub. Date: Oct. 16, 2025

(54) FLUID COLLECTION ASSEMBLIES INCLUDING AT LEAST ONE INFLATION DEVICE AND METHODS AND SYSTEMS OF USING THE SAME

(71) Applicant: PUREWICK CORPORATION, Covington, GA (US)

(72) Inventor: Matthew Jordan Rothberg, Atlanta, GA (US)

(21) Appl. No.: 19/048,004

(22) Filed: Feb. 7, 2025

Related U.S. Application Data
(63) Continuation of application No. 17/663,330, filed on May 13, 2022, now Pat. No. 12,251,333.
(60) Provisional application No. 63/191,558, filed on May 21, 2021.

(51) Int. Cl. A61F 5/455; A61F 5/44; A61M 1/00

(52) U.S. Cl. CPC A61F 5/455; A61F 5/4405; A61M 1/80; A61M 2202/0496; A61M 2210/1092

(57) ABSTRACT

An example fluid collection assembly includes a fluid impermeable barrier defining at least one opening, a chamber, and at least one fluid outlet. The fluid collection assembly also includes at least one porous material disposed in the chamber. The fluid collection assembly further includes at least one inflation device. The inflation device includes bladder having one or more walls defining an interior region. The inflation device also includes at least one valve configured to selectively permit fluid flow into and out of the interior region to switch the bladder between a first state and a second state. The inflation device may at least one of controllably change the length of the fluid collection assembly or controllably change a width of at least a portion of the fluid collection assembly to more comfortably fit the fluid collection assembly within the labia folds of an individual using the fluid collection assembly.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments disclosed herein are directed to fluid collection assemblies that include at least one inflation device, methods for using the same, and systems including the same. In an embodiment, a fluid collection assembly is disclosed. The fluid collection assembly includes a fluid impermeable barrier defining a chamber, at least one opening, and at least one fluid outlet. The fluid collection assembly also includes at least one porous material disposed in the chamber. The fluid collection assembly also includes at least one inflation device including a bladder and at least one valve. The bladder includes one or more walls defining at least one interior region. The at least one valve is configured to selectively permit at least one inflation fluid to flow into and out of the at least one interior region to switch the bladder between a first state and at least a second state. An amount of the at least one inflation fluid present in the at least one interior region is greater when the bladder is in the second state than when the bladder is in the first state. The fluid impermeable barrier exhibits a first length when the bladder exhibits the first state and a second length measure when the bladder exhibits the second state, wherein the first length is less than the second length. The first length and second length measured parallel to a longitudinal axis of the fluid collection assembly.

In an embodiment, a fluid collection assembly is disclosed. The fluid collection assembly includes a fluid impermeable barrier. The fluid impermeable barrier defines a chamber, at least one opening, and at least one fluid outlet. The fluid collection assembly also includes at least one porous material disposed in the chamber. The fluid collection assembly further includes at least one inflation device including a bladder and at least one valve. The bladder includes one or more walls defining at least one interior region. The at least one valve is configured to selectively permit at least one inflation fluid to flow into and out of the at least one interior region to switch the bladder between a first state and at least a second state. An amount of the at least one inflation fluid present in the at least one interior region is greater when the bladder is in the second state than when the bladder is in the first state. The fluid impermeable barrier exhibits a first width measured when the bladder exhibits the first state and a second width when the bladder exhibits the second state. The first width is less than the second width by about 1 cm or less. The first width and the second width measured perpendicular to a longitudinal axis of the fluid collection assembly.

In an embodiment, a fluid collection system is disclosed. The fluid collection system includes a fluid collection assembly. The fluid collection assembly includes a fluid impermeable barrier defining a chamber, at least one opening, and at least one fluid outlet. The fluid collection assembly also includes at least one porous material disposed in the chamber. The fluid collection assembly also includes at least one inflation device including a bladder and at least one valve. The bladder includes one or more walls defining at least one interior region. The at least one valve is configured to selectively permit at least one inflation fluid to flow into and out of the at least one interior region to switch the bladder between a first state and at least a second state. An amount of the at least one inflation fluid present in the at least one interior region is greater when the bladder is in the second state than when the bladder is in the first state. The fluid impermeable barrier exhibits at least one of a first length when the bladder exhibits the first state and a second length measure when the bladder exhibits the second state or a first width measured when the bladder exhibits the first state and a second width when the bladder exhibits the second state. The first length is less than the second length and the first width is less than the second width by about 1 cm or less. The first length and second length measured parallel to a longitudinal axis of the fluid collection assembly and first width and the second width measured perpendicular to a longitudinal axis of the fluid collection assembly. The fluid collection assembly also includes a fluid storage container and a vacuum source. The at least one fluid outlet of the fluid collection assembly, the fluid storage container, and the vacuum source are in fluid communication with each other.

In an embodiment, a method of using a fluid collection assembly is disclosed. The method includes positioning at least one opening of the fluid collection assembly adjacent to a female urethral opening. The fluid collection assembly includes a fluid impermeable barrier including a proximal end region, a distal end region opposite the proximal end region, and two lateral sides extending between the proximal end region and the distal end region. The fluid impermeable barrier defines a chamber, at least one opening between the two lateral sides, and at least one fluid outlet at the proximal end region. The fluid collection assembly also includes at least one porous material disposed in the chamber. The fluid collection assembly further includes at least one inflation device including a bladder and at least one valve. The bladder includes one or more walls defining at least one interior region. The method also includes flowing at least one inflation fluid through the at least one valve and into the at least one interior region of the at least one inflation element to increase at least one of a length of the fluid collection assembly or increase a width of the fluid collection assembly by at most about 1 cm.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly configured to be used to collect bodily fluids from a female urethral opening, according to an embodiment.

FIGS. 1B and 1C are schematic cross-sectional views of the fluid collection assembly taken along plane 1B-1B when a bladder of the fluid collection assembly is in a first state and in a second state, respectively.

FIG. 2A is an isometric view of a fluid collection assembly that includes at least one inflation device adjacent to at least one lateral side of the fluid impermeable barrier, according to an embodiment.

FIGS. 2B and 2C are cross-sectional schematics of the fluid collection assembly taken along plane 2B-2B when the bladder of the inflation device is in the first and second states, respectively.

FIG. 3 is a cross-sectional schematic of a fluid collection assembly that is configured to change a length and a width thereof, according to an embodiment.

FIG. 4 is a cross-sectional schematic of a fluid collection assembly that includes two or more inflation devices in fluid communication with each other, according to an embodiment.

FIG. 5 is a cross-sectional schematic of a fluid collection assembly including a single inflation device that is configured to controllably change the length and width of the fluid collection assembly, according to an embodiment.

FIG. 6 is a block diagram of a system for fluid collection, according to an embodiment.

### DETAILED DESCRIPTION

Embodiments disclosed herein are directed to fluid collection assemblies that include at least one inflation device, methods for using the same, and systems including the same. An example fluid collection assembly includes a fluid impermeable barrier defining at least one opening, a chamber, and at least one fluid outlet. The fluid collection assembly also includes at least one porous material disposed in the chamber. As previously discussed, the fluid collection assembly further includes at least one inflation device. The inflation device includes bladder having one or more walls defining an interior region. The inflation device also includes at least one valve configured to selectively permit fluid flow into and out of the interior region to switch the bladder between a first state (e.g., a deflated and/or initial state) and a second state (e.g., an at least partially inflated state). The inflation device may at least one of controllably change the length of the fluid collection assembly to accommodate different sized vulvas or controllably change the width (e.g., maximum width) of at least a portion of the fluid collection assembly to more comfortably fit the fluid collection assembly within the labia folds of an individual using the fluid collection assembly.

The fluid collection assembly is configured to be positioned about, around, or at least partially within the vulva (e.g., labia majora, labia minora, clitoris, urethral opening, vaginal opening, etc.) of an individual. For example, the fluid collection assembly may be at least partially positioned within or between the labia folds. The fluid collection assembly may also be positioned adjacent to the mons pubis, the perineum, buttocks, and the inner thighs of the individual.

The inflation devices of the fluid collection assemblies disclosed herein allows the fluid collection assemblies to change a size thereof. The ability of the fluid collection assemblies to change a size thereof allows such fluid collection assemblies to be used with a variety of individuals exhibiting different sized anatomy. In particular, the fluid collection assemblies including the at least one inflation device may be used with individuals exhibiting different lengths of vulvas and/or different sizes of labia folds. As used herein, the sizes of the labia folds may refer to the length of one or all of the labia folds, the width of one or all of the labia folds, the thickness of one or all of the labia folds, or, more preferably, the space between two or more of the labia folds in which the fluid collection assembly may be disposed.

Some conventional fluid collection assemblies are unable to change a size thereof which restricts the ability of such conventional fluid collection assemblies to be used effectively with individuals exhibiting different sized anatomies. For example, such conventional fluid collection assemblies may only be used with individuals exhibiting certain sized anatomy. Using such conventional fluid collection assemblies with anatomy that is bigger or smaller than the conventional fluid collection assembly is configured to be used with may result in gaps between the conventional fluid collection assemblies and the vulva. The gaps may allow bodily fluids to leak from the conventional fluid collection assemblies and allow air to enter the chamber of such conventional fluid collection assemblies thereby decreasing the ability of a vacuum pressure to remove bodily fluids from the chamber. Further, such conventional fluid collection assemblies may be uncomfortable to use.

As previously discussed, the inflation devices of the fluid collection assemblies disclosed herein allow the fluid collection assemblies to change a size thereof such that the fluid collection assemblies may be used with different sized anatomy. In an example, the inflation device of the fluid collection assembly may be configured to change a length of the fluid collection assembly. In such an example, the inflation device may be inflated (e.g., at least one inflation fluid may be provided to the inflation device) or deflated (e.g., the inflation fluid may be removed from the inflation device) when the length of the vulva of an individual is greater or less than, respectively, than the length of vulva that the fluid collection assembly is configured to be used with. The ability of the inflation device to increase or decrease the overall length of the fluid collection assembly depending on the length of the vulva of the individual prevents the formation of gaps between the fluid collection assembly and the vulva through which bodily fluids and air may flow. The ability of the inflation device to increase or decrease the overall length of the fluid collection assembly depending on the length of the vulva of the individual may also make the fluid collection assembly more comfortable to use. In an example, the inflation device of the fluid collection assembly may be configured to change a maximum width of the fluid collection assembly. In such an example, the inflation device may be inflated or deflated when the size of the labia folds in which the fluid collection assembly is positioned is greater than or less than the size the labia folds in which the fluid collection assembly is configured to be used with. As such, changing the maximum width of the fluid collection assembly may decrease gaps between the fluid collection assembly and the labia folds and may allow the fluid collection assembly to be positioned closer to the urethral opening of the individual. Further, changing the maximum width of the fluid collection assembly depending on the size of the labia folds allows the fluid collection assembly to be at least partially positioned between the labia folds while maximizing comfort.

It is noted that some conventional fluid collection assemblies may include inflation devices that are configured to be increase the overall width of such conventional fluid collection assemblies such that the conventional fluid collection assemblies contact the thighs of the individual. However, unlike the fluid collection assemblies disclosed herein, such inflation device are not configured to predominately contact and be positioned between the labia folds of the individual since the increase in the width of the fluid collection assemblies required to contact the thighs would make it impossible or uncomfortable to position such a fluid collection assembly between the labia folds.

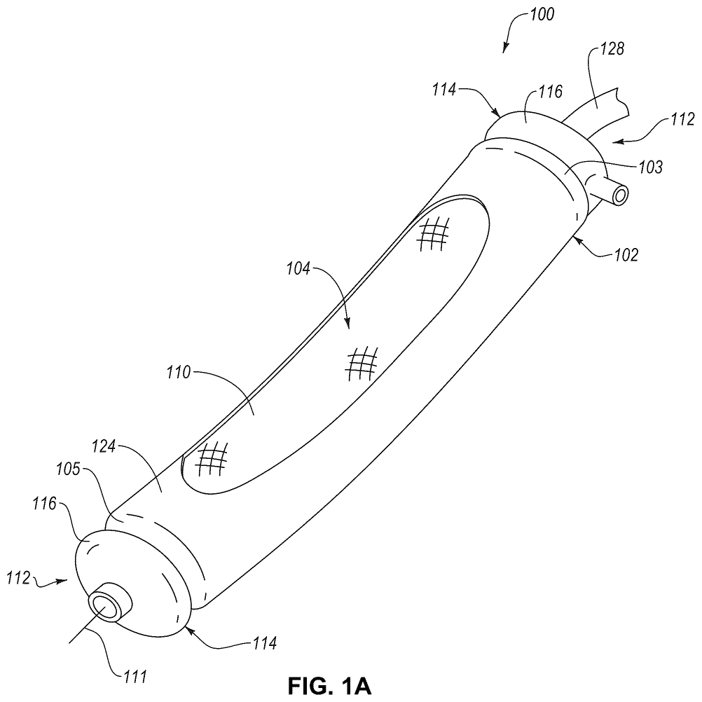

FIG. 1A is an isometric view of a fluid collection assembly 100 configured to be used to collect bodily fluids from a female urethral opening, according to an embodiment. FIGS. 1B and 1C are schematic cross-sectional views of the fluid collection assembly 100 taken along plane 1B-1B when a bladder 114 of the fluid collection assembly 100 is in a first state and in a second state, respectively. The fluid collection assembly 100 includes a fluid impermeable barrier 102 includes a proximal region end 103 and a distal end region 105 opposite the proximal end region 103. The fluid impermeable barrier defines at least one opening 104, a chamber 106, and at least one fluid outlet 108. The fluid outlet 108 may be located at the proximal region end 103. The fluid collection assembly 100 also includes at least one porous material 110 disposed chamber 106. The fluid collection assembly 100 further includes at least one inflation device 112 configured to change a length of the fluid collection assembly 100. The length of the fluid collection assembly 100 is measured parallel to a longitudinal axis 111 of the fluid collection assembly 100.

The inflation device 112 includes a bladder 114. The bladder 114 includes one or more walls 116 defining an interior region 118. The inflation device 112 also includes at least one valve 120 in fluid communication with the interior region 118. The valve 120 is configured to selectively permit flow of at least one inflation fluid into and/or out the interior region 118. For example, the valve 120 may allow the inflation fluid to enter the interior region 118 when it is desirable to at least one of increase the length of the bladder 114 which, in turn, increases the length of the fluid collection assembly 100. The valve 120 may also enable removing the inflation fluid from the interior region 118 when it is desirable to decrease the length (e.g., return to the initial length) of the of the fluid collection assembly 100.

Disposing or removing the inflation fluid into and from the interior region 118 changes the state of the bladder 114. The bladder 114 may exhibit at least a first state and a second state. The amount (volume or weight) of inflation fluid present in the interior region 118 is greater when the bladder 114 is in the second state than when the bladder 114 is in the first state. In an example, as shown in FIG. 1B, the bladder 114 is in the first state when the bladder 114 is in a deflated state (e.g., there are no or substantially no fluids in the interior region 118). However, it is noted that the bladder 114 may be in the first state when some inflation fluid is present in the interior region 118. It is noted that the bladder 114 is illustrated in FIG. 1B as being slightly inflated to facilitate labeling of the interior region 118. The first state of the bladder 114 also generally corresponds to an initial state of the bladder 114 (i.e., generally, the bladder 114 is not provided with the inflation fluid) though, in some examples, the first state of the bladder 114 may not correspond to the initial state of the bladder 114 (e.g., the bladder 114 is provided with fluids). In an example, as shown in FIG. 1C, the bladder 114 is in the second state when the bladder 114 is in an at least partially inflated state. The bladder 114 is generally not in the initial state when the bladder 114 is in the second state though, in some examples, the initial state and the second state of the bladder 114 are the same (e.g., the bladder 114 is provided with fluids).

The bladder 114 may exhibit one or more additional states (e.g., third state, fourth state, and so forth) besides the first and second states discussed above. In an embodiment, the one or more additional states may include less of the inflation fluid in the interior region 118 (e.g., is more deflated) than the first state (e.g., the first state is a partially inflated state). In such an embodiment, the one or more additional states may include a deflated or partially deflated state and may be formed by removing the inflation fluid from the interior region 118 when the bladder 114 is in the first or second state. In an embodiment, the one or more additional states may include more of the inflation fluid in the interior region 118 (e.g., is more inflated) than the first state (e.g., the first state is a deflated or partially inflated state) and include less of the inflation fluid in the interior region 118 than the second state. In such an embodiment, the one or more additional states include a partially inflated state and may be formed by adding the inflation fluid to or removing the inflation fluid from the interior region 118 when the bladder 114 is in the first state or second state, respectively. In an embodiment, the one or more additional states may include more of the inflation fluid in the interior region 118 than the second state (e.g., the second state is a partially inflated state). In such an embodiment, the one or more additional states may be an at least partially inflated state and may be formed by adding the inflation fluid to the interior region 118 when the bladder 114 is in the first state or second state. It is noted that, in some embodiments, the bladder 114 may only include the first and second states.

As shown in FIGS. 1B and 1C, switching the bladder 114 from the first state to the second state (or any of the other states thereof) changes the shape of the bladder 114 and/or increases a distance that a portion of the bladder 114 extends from the fluid impermeable barrier 102 thereby changing the length of the fluid collection assembly 100. For example, referring to FIG. 1B, the fluid collection assembly 100 may exhibit a shape that generally conforms to the shape of the portion of the fluid impermeable barrier 102 to which the bladder 114 is attached when the bladder 114 is in the first state. The fluid collection assembly 100 may exhibit a first length L₁ when the bladder 114 is in the first state. Switching the bladder 114 to the second state changes the shape of the fluid collection assembly 100, as shown in FIG. 1C, to a shape that does not conform to the shape of the fluid impermeable barrier 102 to which the bladder 114 is attached. Further, switching the bladder 114 from the first state to the second state increases the distance that a portion of the bladder 114 extends from the fluid impermeable barrier 102 thereby increasing the length of the fluid collection assembly 100. For instance, switching the bladder 114 from the first state to the second state increases the length of the fluid collection assembly 100 to the second length L₂ which is greater than the first length L₁. The increased length of the fluid collection assembly 100 may allow the fluid collection assembly 100 to be used with longer vulvas without forming gaps between the fluid collection assembly 100 and the vulva through which bodily fluids and air may flow. It is noted that the inflation fluid may be removed from the interior region 118 using the valve 120, for example, when too much of the inflation fluid is added to the interior region 118 which may at least one of increase the risk that the bladder 114 ruptures, make the fluid collection assembly 100 more uncomfortable, or increase the likelihood that the fluid collection assembly 100 leaks bodily fluids since the fluid collection assembly 100 exhibits a length that is too great for the vulva.

The walls 116 are formed from a material is substantially impermeable to the inflation fluid (e.g., substantially impermeable to a gas and/or a liquid) which allows the bladder 114 to retain the inflation fluid without embarrassing leaks. The walls 116 may also be formed from a flexible material. The flexible material of the walls 116 allows the bladder 114 and, by extension, the fluid collection assembly 100 to at least one of increase in size or change a shape thereof. For example, the flexible material of the walls 116 allow the interior region 118 to increase a volume thereof when the interior region 118 receives an inflation fluid and decrease a volume thereof when the inflation fluid is removed from the interior region 118. Examples of materials that may form the walls 116 of the bladder 114 include silicone, rubber, latex, polychloroprene, nylon fabric, polypropylene, polyvinyl chloride, nitrile rubber, other suitable polymers, a metal foil, a composite, or combinations thereof. In an embodiment, the walls 116 are configured to stretch (e.g., elastically or plastically stretch) so the walls 116 remain taut when the bladder 114 is at least partially inflated. In an embodiment, the wall 116 forms a plurality of wrinkles when the bladder 114 is at least partially deflated and adding inflation fluid into the interior region 118 decreases the wrinkles, similar to an accordion.

The bladder 114 of the inflation device 112 and, in particular, the walls 116 of the bladder 114 may exhibit any suitable shape. In an embodiment, as previously discussed, the bladder 114 may exhibit a shape that generally corresponds to the shape of the region of the fluid impermeable barrier 102 that the bladder 114 is adjacent to when the bladder 114 is in the deflated state. In such an embodiment, the bladder 114 may exhibit a shape that is different than the region of the fluid impermeable barrier 102 that the bladder 114 is adjacent to when the bladder 114 is in at least partially inflated state. In an embodiment, the fluid collection assembly 100 may include a bladder 114 adjacent to the proximal region end 103 of the fluid impermeable barrier 102. In such an embodiment, the bladder 114 may exhibit a generally annular shape, a generally C-shape, or another suitable shape (e.g., a shape defining a hole) which allows the bladder 114 to not obstruct the fluid outlet 108.

The valve 120 may include any suitable valve configured to allow for the controllable addition and remove of the inflation fluid from the interior region 118. In an embodiment, the valve 120 is a luer valve and includes a male-tapper fitting or a female-taper fitting. In an embodiment, the valve 120 includes a fluid impermeable membrane with a slit or opening formed. The slit or opening of the fluid impermeable membrane remains substantially closed when no external load is applied thereto but opens when an external load is applied thereto (e.g., an external load caused by pressing a syringe against the fluid impermeable membrane). In an embodiment, the valve 120 may include a mechanical valve, such as a ball valve, a butterfly valve, or any other suitable mechanical valve. The mechanical valve may be manually operated or controlled using a computer. In an embodiment, the valve 120 may include a check valve to limit leaks from the bladder 114 and to make the fluid collection assembly 100 easier to use. In such an embodiment, the valve 120 may only add or remove (but not both) inflation fluid from the interior region 118 and, as such, the fluid collection assembly 100 is configured for single use.

In an embodiment, the valve 120 extends from or near the proximal region end 103, the distal end region 105, or a back side 122 of the fluid impermeable barrier 102. The back side 122 of the fluid impermeable barrier 102 is the side of the fluid impermeable barrier 102 that is generally opposite the opening 104. The valve 120 at or near the proximal region end 103, the distal end region 105, or a back side 122 may allow a user of the fluid collection assembly 100 (e.g., medical practitioner, nurse, or the individual using the fluid collection assembly 100) to access the valve 120 when the fluid collection assembly 100 is adjacent to the vulva since, generally, the inner thighs of the individual may contact or obstruct the surfaces of the fluid impermeable barrier 102 except the proximal region end 103, the distal end region 105, or a back side 122. Further, the valve 120 at or near the proximal region end 103, the distal end region 105, or a back side 122 prevents the valve 120 from pressing against the inner thighs during use which may cause discomfort.

In an embodiment, the fluid collection assembly 100 may only include a single inflation device 112 (e.g., a single bladder 114 and/or a single valve 120). For example, the fluid collection assembly 100 may only include the inflation device 112 at the proximal region end 103 or distal end region 105. In an embodiment, the fluid collection assembly 100 may include a plurality of inflation device 112 (e.g., a plurality of bladders 114) and/or a plurality of valves 120. For example, as illustrated, the fluid collection assembly 100 may include two inflation devices 112. One of the inflation device 112 may be attached to or otherwise proximate to the proximal region end 103 of the fluid impermeable barrier 102 and the other inflation device 112 may be attached to or otherwise proximate to the distal end region 105 of the fluid impermeable barrier 102. The two inflation devices 112 attached to the proximal and distal end regions 103, 105 allow the length of the fluid collection assembly 100 to increase from either or both of the proximal or distal end regions 103, 105 thereof. Increasing the length of the fluid collection assembly 100 from one or both of the proximal or distal end regions 103, 105 allows better control over the position of the fluid collection assembly 100 such that the opening 104 is adjacent to the urethral opening of the individual and allows for the fluid collection assembly to better fit the vulva. Each inflation device 112 (e.g. each bladder 114) may include one or more valves 120 to allow for independent inflation of the bladders 114 which allows for better control of the shape and size of the fluid collection assembly 100. However, the fluid collection assembly 100 may only include a single valve 120 for two or more bladders 114 when the two or more bladders 114 are fluidly coupled together using one or more tubes extending therebetween. In some examples, a single bladder 114 may include a plurality of valves 120, for instance, to increase the likelihood that one valve 120 is easily accessible.

The inflation device 112 may include additional components other than the components discussed above. For example, the inflation device 112 may include less flexible material (e.g., rigid material or material that is less flexible than the walls 116) that are used to control the shape of the bladder 114 when the bladder 114 is inflated (e.g., switched from the first state to the second state). The less flexible material may form parts of the walls 116, wrap around the walls 116, or extend in the interior region 118 between opposing portions of the walls 116 which limits expansion of the walls 116 at and near the less flexible material.

The at least one inflation fluid added or removed from the interior region 118 may include any suitable fluid, such as any suitable liquid or any suitable gas. In an embodiment, the inflation fluid is formed from at least one generally regarded as safe (“GRAS”) material. Forming the inflation fluid from a GRAS material may decrease health risks caused by inadvertently exposing the individual to the inflation fluid. Examples of GRAS materials that may form the inflation fluid includes water, saline solution, alcohol solution, atmospheric air, nitrogen, or combinations thereof.

As previously discussed, the fluid collection assembly 100 includes a fluid impermeable barrier 102. In an embodiment, as shown, the fluid impermeable barrier 102 forms at least a portion of the inflation device 112 since the fluid impermeable barrier 102 defines a portion of the interior region 118. In such an embodiment, the walls 116 of the bladder 114 are attached to the fluid impermeable barrier 102 in a substantially fluid tight manner to prevent the inflation fluid leaking from the interior region 118. In an embodiment, the bladder 114 is distinct from the fluid impermeable barrier 102 (e.g., the fluid impermeable barrier 102 does not partially define the interior region 118). In such an embodiment, the wall 116 of the bladder 114 may be attached to the fluid impermeable barrier 102. In an embodiment, the bladder 114 is spaced from the fluid impermeable barrier 102, such as when the bladder 114 is disposed within the chamber 106. Examples of other locations of the bladder 114 relative to the fluid impermeable barrier 102 are disclosed in U.S. Provisional Patent Application No. 63/030,685 filed on May 27, 2020, the disclosure of which is incorporated herein, in its entirety, by this reference.

The fluid impermeable barrier 102 may be formed of any suitable fluid imporous material(s), such as a fluid impermeable polymer (e.g., silicone, polypropylene, polyethylene, polyethylene terephthalate, a polycarbonate, etc.), a metal film, natural rubber, another suitable material, or combinations thereof. The fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least a surface of the fluid impermeable barrier 102 that may contact the individual may be formed from a soft and/or smooth material (e.g., silicone), thereby reducing chaffing. In an embodiment, the fluid impermeable barrier 102 may be formed from a flexible material, such as silicone, which allows the fluid impermeable barrier 102 to be bent into a shape that conforms the anatomy of the individual. Further, as shown in FIGS. 1B and 1C, forming the fluid impermeable barrier 102 from a flexible material allows the fluid impermeable barrier 102 to accommodate the shape and/or size changes by switching the fluid collection assembly 100 and the bladder 114 between states.

In some examples, the fluid impermeable barrier 102 may be tubular (ignoring the opening), such as substantially cylindrical (as shown), oblong, prismatic, or flattened tubes when the bladder 114 is in the first state and/or the second state. During use, the outer surface 124 of the fluid impermeable barrier 102 may contact the individual. The fluid impermeable barrier 102 may be sized and shaped to fit in the gluteal cleft between the legs of a female user when the bladder 114 are in at least the second state.

The opening 104 provides an ingress route for fluids to enter the chamber 106. The opening 104 may be defined by the fluid impermeable barrier 102 such as by an inner edge of the fluid impermeable barrier 102. For example, the opening 104 is formed in and extends through the fluid impermeable barrier 102, from the outer surface 124 to the inner surface 126, thereby enabling fluid(s) to enter the chamber 106 from outside of the fluid collection assembly 100. The opening 104 may be an elongated hole in the fluid impermeable barrier 102. For example, the opening 104 may be defined as a cut-out in the fluid impermeable barrier 102. The opening 104 may be located and shaped to be positioned adjacent to a female urethra.

The fluid collection assembly 100 may be positioned proximate to the female urethral opening and urine may enter the chamber of the fluid collection assembly 100 via the opening 104. The fluid collection assembly 100 is configured to receive the bodily fluids into the chamber 106 via the opening 104. When in use, the opening 104 may have an elongated shape that extends from a first location below the urethral opening (e.g., at or near the anus or the vaginal opening) to a second location above the urethral opening (e.g., at or near the top of the vaginal opening or the pubic hair).

The opening 104 may have an elongated shape because the space between the legs of a female is relatively small when the legs of the female are closed, thereby only permitting the flow of the fluid(s) along a path that corresponds to the elongated shape of the opening 104 (e.g., longitudinally extending opening). The opening 104 in the fluid impermeable barrier 102 may exhibit a length measured along the longitudinal axis 111 of the fluid collection assembly 100 that may be at least about 10% of the length of the fluid collection assembly 100, such as about 25% to about 50%, about 40% to about 60%, about 50% to about 75%, about 65% to about 85%, or about 75% to about 95% of the length of the fluid collection assembly 100.

The opening 104 in the fluid impermeable barrier 102 may exhibit a width measured transverse to the longitudinal axis 111 of the fluid collection assembly 100 that may be, when the fluid collection assembly 100 and the bladder 114 are in the first state, at least about 10% of the circumference of the fluid collection assembly 100, such as about 25% to about 50%, about 40% to about 60%, about 50% to about 75%, about 65% to about 85%, or about 75% to about 100% of the circumference of the fluid collection assembly 100. The opening 104 may exhibit a width that is greater than 50% of the circumference of the fluid collection assembly 100 since the vacuum (e.g., suction) through the conduit 128 pulls the fluid through the porous material 110 and into the conduit 128. As shown in FIGS. 1B and 1C, switching the bladder 114 from the first state to the second state increases the width of the opening 104. The increased width of the opening 104 may allow the opening 104 to receive more bodily fluids than if the opening 104 did not exhibit the increased width.

In some examples, the opening 104 may be vertically oriented (e.g., having a major axis parallel to the longitudinal axis 111 of the fluid collection assembly 100). In some examples (not shown), the opening 104 may be horizontally oriented (e.g., having a major axis perpendicular to the longitudinal axis 111 of the fluid collection assembly 100). In an example, the fluid impermeable barrier 102 may be configured to be attached to the individual, such as adhesively attached (e.g., with a hydrogel adhesive) to the individual. According to an example, a suitable adhesive is a hydrogel layer.

As previously discussed, the fluid impermeable barrier 102 may define fluid outlet 108 configured to remove bodily fluids from the chamber 106. The fluid outlet 108 is distinct from the opening 104 and the valve 120. In some examples, the fluid outlet 108 is sized to receive the conduit 128. The conduit 128 may be disposed in the chamber 106 via the fluid outlet 108. The fluid outlet 108 may be sized and shaped to form an at least substantially fluid tight seal against the conduit 128 or the at least one tube substantially preventing the bodily fluids from escaping the chamber 106.

The fluid impermeable barrier 102 may include markings thereon, such as one or more markings to aid a user in aligning the fluid collection assembly 100 on the individual. For example, a line on the fluid impermeable barrier 102 (e.g., opposite the opening 104) may allow a healthcare professional to align the opening 104 over the urethral opening of the individual. In examples, the markings may include one or more of alignment guide or an orientation indicator, such as a stripe or hashes. Such markings may be positioned to align the fluid collection assembly 100 to one or more anatomical features such as a pubic bone, etc.

As previously discussed, the fluid collection assembly 100 includes porous material 110 disposed in the chamber 106. The porous material 110 may cover at least a portion (e.g., all) of the opening 104. The porous material 110 is exposed to the environment outside of the chamber 106 through the opening 104. The permeable properties referred to herein may be wicking, capillary action, absorption, diffusion, or other similar properties or processes, and are referred to herein as “permeable” and/or “porous.” The porous material 110 may also wick the bodily fluids generally towards an interior of the chamber 106, as discussed in more detail below. The porous material 110 may include one or more of a fluid permeable membrane 130 or a fluid permeable support 132.

In an embodiment, at least a portion of the porous material 110 may be a wicking material configured to wick and/or allow flow of the bodily fluids away from the opening 104, thereby preventing bodily fluids from escaping the chamber 106. The wicking material may not include absorption of the bodily fluids into the wicking material. Put another way, substantially no absorption of the bodily fluids into the wicking material may take place after the wicking material is exposed to the bodily fluids. While no absorption is desired, the term “substantially no absorption” may allow for nominal amounts of absorption of the bodily fluids into the wicking material (e.g., absorbency), such as about 10 wt % of the dry weight of the wicking material, about 7 wt %, about 5 wt %, about 3 wt %, about 2 wt %, about 1 wt %, or about 0.5 wt % of the dry weight of the wicking material.

The fluid collection assembly 100 may include the fluid permeable membrane 130 disposed in the chamber 106. The fluid permeable membrane 130 may cover at least a portion (e.g., all) of the opening 104. The fluid permeable membrane 130 may be composed to pull/push the bodily fluids away from the opening 104, thereby promoting fluid flow into the chamber 106, prevent fluid remaining on the vulva of the individual, and preventing the bodily fluids from escaping the chamber 106.

The fluid permeable membrane 130 may include any material that may be permeable to the bodily fluids. For example, the fluid permeable membrane 130 may include fabric, such as a gauze (e.g., a silk, linen, or cotton gauze), another soft fabric, or another smooth fabric. Forming the fluid permeable membrane 130 from gauze, soft fabric, and/or smooth fabric may reduce chaffing caused by the fluid collection assembly 100 and makes wearing the fluid collection assembly more comfortable. In an embodiment, the fluid permeable membrane 130 is formed from a flexible material, such as gauze, since the shape and/or size of the fluid permeable membrane 130 may change when the fluid collection assembly 100 and the bladder 114 switch between states, as shown in FIGS. 1B and 1C. In an embodiment, the fluid permeable membrane 130 may define a plurality of perforations or may be continuous (e.g., does not define perforations). In an embodiment, the fluid permeable membrane 130 defines at least one hole that is configured to allow the valve 120 to extend through the fluid permeable membrane 130.

The fluid collection assembly 100 may include the fluid permeable support 132 disposed in the chamber 106. The fluid permeable support 132 is configured to support the fluid permeable membrane 130 and maintain the shape of the chamber 106 since the fluid impermeable barrier 102 and the fluid permeable membrane 130 may be formed from a relatively foldable, flimsy, or otherwise easily deformable material. For example, the fluid permeable support 132 may be positioned so the fluid permeable membrane 130 is disposed between the fluid permeable support 132 and the fluid impermeable barrier 102. The fluid permeable support 132 may support and maintain the position of the fluid permeable membrane 130 and the shape of the chamber 106. The fluid permeable support 132 may include any material that may be permeable to the bodily fluids, such as any of the fluid permeable membrane 130 materials disclosed above. For example, the fluid permeable membrane 130 material(s) may be utilized in a more dense or rigid form than in the fluid permeable membrane 130 when used as the fluid permeable support 132. The fluid permeable support 132 may be formed from any fluid porous material that is less deformable than the fluid permeable membrane 130. For example, the fluid permeable support 132 may include a porous polymer (e.g., nylon, polyester, polyurethane, polyethylene, polypropylene, etc.) structure (e.g., spun fibers such as spun nylon fibers) or a foam (e.g., an open cell foam). In some examples, the fluid permeable support 132 may be formed from a natural material, such as cotton, wool, silk, or combinations thereof. In such examples, the material may have a coating to prevent or limit absorption of the bodily fluids into the material, such as a water repellent coating. In some examples, the fluid permeable support 132 may be formed from fabric, felt, gauze, or combinations thereof.

In some examples, the fluid permeable membrane 130 may be optional. For example, the porous material 110 may include only the fluid permeable support 132. In such examples, the bladder 114 may be positioned within the fluid permeable support 132 since, for instance, at least some materials of the support 132 disclosed herein are flexible enough to accommodate the shape and/or size changes discussed herein. In some examples, the fluid permeable support 132 may be optionally omitted from the fluid collection assembly 100 and the porous material 110 may only include the fluid permeable membrane 130. In such examples, the bladder 114 may be positioned within the fluid permeable membrane 130.

In an embodiment, the fluid permeable membrane 130 and/or the fluid permeable support 132 are wicking materials. In such an embodiment, the fluid permeable support 132 may have a greater ability to wick the bodily fluids than the fluid permeable membrane 130, such as to move the bodily fluids inwardly from the 116 of the fluid collection assembly 100. In some examples, the wicking ability of the fluid permeable support 132 and the fluid permeable membrane 130 may be substantially the same. In an embodiment, the fluid permeable membrane 130 and/or the fluid permeable support 132 are non-wicking materials (e.g., absorbent materials).

In an embodiment, not shown, the fluid permeable membrane 130 and the fluid permeable support 132 may at least substantially completely fill the portions of the chamber 106 not occupied by the inflation device 112 and the conduit 128. In an embodiment, as shown in FIGS. 1B and 1C, the fluid permeable membrane 130 and the fluid permeable support 132 may not substantially completely fill the portions of the chamber 106 not occupied by the inflation device 112 or the conduit 128. In such an embodiment, the fluid collection assembly 100 includes the fluid reservoir 134 disposed in the chamber 106.

The fluid reservoir 134 is a substantially unoccupied portion of the chamber 106. The fluid reservoir 134 may be defined between the fluid impermeable barrier 102 and at least one of the inflation device 112, the fluid permeable membrane 130, or the fluid permeable support 132. The bodily fluids in the chamber 106 may flow through the fluid permeable membrane 130 and/or fluid permeable support 132 to the fluid reservoir 134. The fluid reservoir 134 may retain of the bodily fluids. The bodily fluids in the chamber 106 may flow through the fluid permeable membrane 130 and/or fluid permeable support 132 and, optionally, to the fluid reservoir 134. The fluid impermeable barrier 102 may retain the bodily fluids in the fluid reservoir 134. The fluid reservoir 134 may be in a portion of the chamber 106 designed to be in a gravimetrically low point of the fluid collection assembly 100 when the fluid collection assembly 100 is worn.

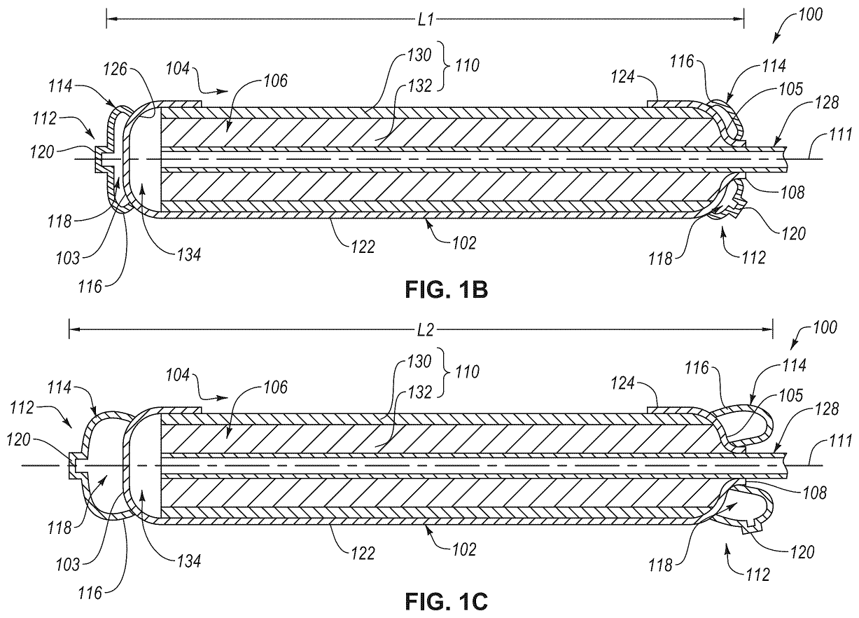

The inflation devices and, in particular, the bladders disclosed herein may be located adjacent to regions of the fluid collection assemblies other than or in addition to the proximal end region and the distal end region of the fluid impermeable barrier. For example, FIG. 2A is an isometric view of a fluid collection assembly 200 that includes at least one inflation device 212 adjacent to at least one lateral side 236 of the fluid impermeable barrier 202, according to an embodiment. FIGS. 2B and 2C are cross-sectional schematics of the fluid collection assembly 200 taken along plane 2B-2B when the bladder 214 of the inflation device 212 is in the first and second states, respectively. The inflation device 212 adjacent to the lateral side 236 of the fluid impermeable barrier 202 allows a width of the fluid collection assembly 200 to be selectively and controllably changed. The width of the fluid collection assembly 200 is measured perpendicular to a longitudinal axis 211 of the fluid collection assembly 200. Except as otherwise disclosed herein, the fluid collection assembly 200 is the same or substantially similar to any of the fluid collection assemblies disclosed herein. For example, the fluid collection assembly 200 may include a fluid impermeable barrier 202 defining at least one opening 204, a chamber 206, and a fluid outlet 208. As previously discussed, the fluid collection assembly 200 also includes at least one inflation device 212. The inflation device 212 includes a bladder 214 having one or more walls 216 defining an interior region 218 and at least one valve 220.

The fluid collection assembly 200 is configured to be at least partially positioned between the labia folds of an individual. Positioning the fluid collection assembly 200 at least partially between the labia folds may decrease the number and/or size of gaps formed between the fluid collection assembly 200 and the vulva thereby minimizing leakage of the bodily fluids and the suction pressure between the fluid collection assembly 200 and the vulva. Also, positioning the fluid collection assembly 200 at least a portion between the labia folds may decrease the distance between the urethral opening of the individual and the fluid collection assembly 200 which increases the quantity of bodily fluids that are discharged from the urethral opening that are received by the fluid collection assembly 200 (e.g., received through the opening 204 and into the porous material 210). As previously discussed, the size of the labia folds may vary from individual to individual. Fluid collection assemblies exhibiting a maximum width that is too large for the particular size of the labia folds may make positioning such a fluid collection assembly between the labia folds difficult, uncomfortable, and may limit the ability to position such a wide fluid collection assembly proximate to the urethral opening of the individual. Fluid collection assembly exhibiting a maximum width that is too small for the particular size of the labia folds may form gaps between the narrow fluid collection assembly and the labia folds through which the bodily fluids and the suction pressure may leak.

The fluid collection assembly 200 is configured to change a width of at least a portion thereof thereby allowing the fluid collection assembly 200 to be used effectively with individuals with differently sized labia folds. In particular, the inflation device 212 allows the width of the fluid collection assembly 200 to be selectively and controllably changed thereby allowing the width of at least a portion of the fluid collection assembly 200 to be controllably and selectively changed. As previously discussed, the inflation device 212 may be disposed adjacent to the at least one lateral side 236 of the fluid impermeable barrier 202 which allows the inflation device 212 to control the width of at least a portion of the fluid collection assembly 200. The lateral side 236 of the fluid impermeable barrier 202 may include a side of the fluid impermeable barrier 202 that is between the opening 204 and the back side 222 of the fluid impermeable barrier 202 and between the proximal and distal end regions 203, 205 of the fluid impermeable barrier 202. As shown in FIG. 2B, a portion of the fluid collection assembly 200 may exhibit a first width W₁ when the bladder 214 is in the first (e.g., deflated) state. The fluid collection assembly 200 may be used with relatively small sized labia folds when the fluid collection assembly 200 exhibits the first width W₁ (e.g., when the bladder 214 is in the first state). However, the fluid collection assembly 200 may also be used with relatively large sized labia folds (e.g., labia folds that are larger than the relatively small sized labia folds). When used with the relatively large sized labia folds, the at least one inflation fluid may be disposed in the interior region 218 of the bladder 214 via the valve 220 to switch the bladder 214 to the second state thereof. As shown in FIG. 2C, switching the bladder 214 from the first state to the second state increases the width of the portion of the fluid collection assembly 200 from the first width W₁ to a second width W₂ that is greater than the first width W₁. As such, the fluid collection assembly 200 may be used more effectively with the relatively large sized labia folds when the fluid collection assembly 200 exhibits the second width W₂ than when the fluid collection assembly 200 exhibits the first width W₁.

At least some convention fluid collection assemblies exhibit a maximum width that is greater than 3 cm. Such widths of the conventional fluid collection assemblies allows the conventional fluid collection assemblies to be used with a significant number (but not all) individuals. In an embodiment, the fluid collection assembly 200 may exhibit a maximum width that is about 3 cm or greater when the bladder 214 exhibits the deflated state. In an embodiment, the fluid collection assembly 200 may exhibit a maximum width when the bladder 214 exhibits the deflated state that is about 3 cm or less, such as about 2.75 cm or less, about 2.5 cm or less, about 2.25 cm or less, about 2 cm or less, about 1.75 cm or less, about 1.5 cm or less, or in ranges of about 1.5 cm to about 2 cm, about 1.75 cm to about 2.25 cm, about 2 cm to about 2.5 cm, about 2.25 cm to about 2.75 cm, or about 2.5 cm to about 3 cm. Selecting the maximum width of the fluid collection assembly 200 to be about 3 cm or less may decrease the volume of the chamber 206 which limits the quantity of bodily fluids that may be stored in the chamber 206 but also allows the fluid collection assembly 200 to be used with individuals having relatively small sized labia folds. Further, the inflation device 212 allows the fluid collection assembly 200 to be used with individuals having relatively large sized labia folds by switching the bladder 214 from the first state to the second state even when the fluid collection assembly 200 exhibits a maximum width that is less than 3 cm.

In an embodiment, the at least one inflation device 212 may be configured such that the maximum difference between the second width W₂ is greater than the first width W₁ by about 2 cm or less, about 1.75 cm or less, about 1.5 cm or less, about 1.25 cm or less, about 1 cm or less, about 7.5 mm or less, about 6 mm or less, about 5 mm or less, about 4 mm or less, about 3 mm or less, about 2 mm or less, about 1 mm or less, or in ranges of about 1 mm to about 3 mm, about 2 mm to about 4 mm, about 3 mm to about 5 mm, about 4 mm to about 6 mm, about 5 mm to about 7.5 mm, about 6 mm to about 1 cm, about 7.5 mm to about 1.25 cm, about 1 cm to about 1.5 cm, about 1.25 cm to about 1.75 cm, or about 1.5 cm to about 2 cm. For example, the difference in sizes of the labia folds from a relatively small sized labia folds to a relatively large sized labia folds may only vary by at most 2 cm and, more commonly, less than 1.5 cm and less than 1 cm. Restricting the maximum change in the width that the portion of the fluid collection assembly 200 may exhibit may prevent overinflation of the bladder 214 when the fluid collection assembly 200 is positioned between the labia folds since overinflation may injure the labia folds. In an embodiment, the fluid collection assembly 200 may include a rigid band extending around at least a portion of or integrally formed with the inflation device 212 that prevents overinflation of the bladder 214.

In an embodiment, the inflation device 212 (e.g., the bladder 214) is positioned adjacent to a portion of the lateral side 236 of the fluid impermeable barrier 202 that is closer to the opening 204 than the back side 222 of the fluid impermeable barrier 202. The inflation device 212 may exhibit such a location since the portion of the lateral sides 236 that is closer to the opening 204 than the back side 222 is more likely to be positioned between the labia folds of the individual than a portion of the lateral side 236 that is closer to the back side 222 than the opening 204. As such, positioning the inflation device 212 to be closer to the opening 204 than the back side 222 better ensures that the inflation device 212 (e.g., the bladder 214) is positioned between the labia folds and that the bladder 214 is more likely to contact the labia folds than the thighs of the individual. In an embodiment, the inflation device 212 may be positioned adjacent to a portion of the lateral side 236 that is equally spaced from the opening 204 and the back side 222 or closer to the back side 222 than the opening 204.

In an embodiment, positioning the inflation device 212 to be closer to the opening 204 than the back side 222 allows the fluid collection assembly 200 to include one or more additional inflation devices (not shown) that are distinct and separate from the inflation devices 212. The additional inflation devices may be positioned adjacent to a portion of the lateral sides 236 that is closer to the back side 222 than the opening 204. The additional inflation devices may be configured to contact the thighs of the patient instead of the labia folds of the individual since the additional inflation devices are positioned closer to the back side 222 which decreases the likelihood that the addition inflation devices contact the labia folds. The additional inflation devices may be configured to increase the width of the fluid collection assembly 200 more than the inflation devices 212.

In an embodiment, the fluid collection assembly 200 includes a plurality of inflation devices 212 (e.g., plurality of bladders 214). In such an embodiment, at least one of the inflation devices 212 may be positioned on one side of the opening 204 and at least one other inflation device 212 may be positioned on the other side of the opening 204. Positioning the inflation devices 212 on both sides of the opening 204 allows for better control of the position of the opening 204 relatively to the urethral opening. For example, positioning the inflation devices 212 on both sides of the opening 204 may allow the central portion of the opening 204 to be positioned adjacent to the urethral opening of the individual by controllably inflating and/or deflating the bladders 214 on one or both sides of the opening 204. Positioning the central portion of the opening 204 adjacent to the urethral opening may maximize the quantity of bodily fluids that are received into the fluid collection assembly 200.

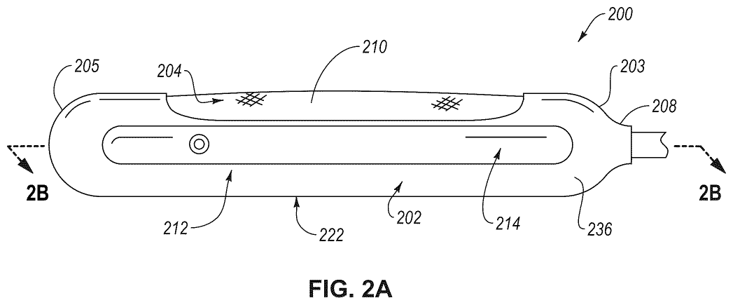

The fluid collection assemblies illustrated in FIGS. 1A to 2C are only configured to controllably and selectively change a length or a width thereof. However, any of the fluid collection assemblies disclosed herein may be configured to change both the length and the width thereof. For example, FIG. 3 is a cross-sectional schematic of a fluid collection assembly 300 that is configured to change a length and a width thereof, according to an embodiment. Except as otherwise disclosed herein, the fluid collection assembly 300 is the same or substantially similar to any of the fluid collection assemblies disclosed herein.

The fluid collection assembly 300 includes a fluid impermeable barrier 302 that includes a proximal end region 303, a distal end region 305 opposite the proximal end region 303, and at least one lateral side 336. The fluid collection assembly 300 includes at least one first inflation device 312a (e.g., at least one first inflation device 312a adjacent to the proximal end region 303 and at least one other inflation device 312a adjacent to the distal end region 305) that is configured to controllably change the length of the fluid collection assembly 300. For example, the first inflation device 312a may be the same or substantially similar to the inflation device(s) 112 illustrated in FIGS. 1A-1C. The fluid collection assembly 300 also includes at least one second inflation device 312b (e.g., at least one second inflation device 312b on one side of the opening (not shown) and at least one other second inflation device 312b on the other side of the opening) on the at least one lateral side 336 of the fluid impermeable barrier 302. The second inflation device 312b is configured to controllably change the width of at least a portion of the fluid collection assembly 300 and, as such, the second inflation device 312b may be the same or substantially similar to the inflation device(s) 212 illustrated in FIGS. 2A-2C. Thus, the first and second inflation devices 312a, 312b allow the fluid collection assembly 300 to controllably change the length and the width thereof thereby allowing the fluid collection assembly 300 to be configured to be used with a wider variety of individuals regardless of the length of the vulva and the size of the labia folds of the individual.

The at least one first inflation device 312a and the at least one second inflation device 312b are distinct and separate from each other. Further, the first inflation device 312a and the second inflation devices 312b are not in fluid communication with each other. This allows the first and second inflation devices 312a, 312b to be independently switched from the first state to second state and vice versa (e.g., independently inflated and deflated). Independently switching the first and second inflation devices 312a, 312b allows for better control of the size of the fluid collection assembly 300. For example, the first inflation device 312a may be inflated when the vulva of the individual is relatively long but the second inflation device 312b may remain deflated when the labia folds of the individual are relatively small.

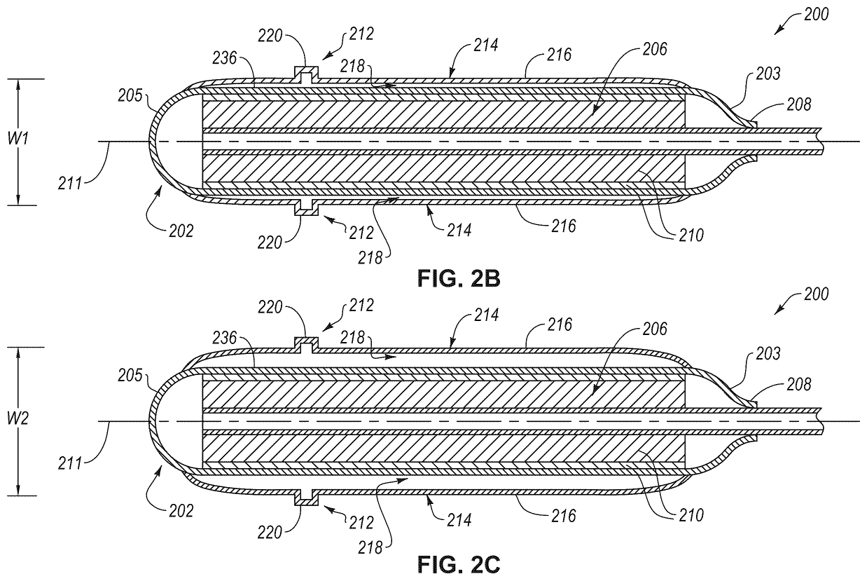

The first and second inflation devices 312a, 312b may be in fluid communication with each other. For example, FIG. 4 is a cross-sectional schematic of a fluid collection assembly 400 that includes two or more inflation devices in fluid communication with each other, according to an embodiment. Except as otherwise disclosed herein, the fluid collection assembly 400 is the same or substantially similar to any of the fluid collection assemblies disclosed herein.

The fluid collection assembly 400 includes a fluid impermeable barrier 402 that includes a proximal end region 403, a distal end region 405 opposite the proximal end region 403, and at least one lateral side 436. The fluid collection assembly 400 includes at least one first inflation device 412a that is configured to controllably change the length of the fluid collection assembly 400. The first inflation device 412a may include, for example, at least one first inflation device 412a adjacent to the proximal end region 403 and/or at least one other first inflation device 412a adjacent to the distal end region 405. The fluid collection assembly also includes at least one second inflation device 412b configured to control the width of at least a portion of the fluid collection assembly 400. The second inflation device 412b may include at least one second inflation device 412b on one side of the opening (not shown) and/or at least one other second inflation device 412b on the other side of the opening.

At least two of the inflation devices of the fluid collection assembly 400 may be in fluid communication with each other. The inflation devices of the fluid collection assembly 400 may be in fluid communication with each other when the inflation fluid in one inflation device may flow to another inflation device through at least one tube 438. The tube 438 may include any structure that defines a passageway that allows the inflation fluid to flow therein and substantially prevents the inflation fluid from leaving the passageway. The tube 438 allows inflating one inflation device to also inflate at least one other inflation device and deflating one inflation to also deflate at least one other inflation device. Configuring at least two of the inflation devices to be in fluid communication with each other may help improve the comfort of using the fluid collection assembly 400 and prevent rupturing the bladders thereof when the individual using the fluid collection assembly 400 moves. For example, when the individual using the fluid collection assembly 400 moves, the individual may compress a portion of the bladder of one of the plurality of inflation devices. When the inflation device is not in fluid communication with another inflation device, the inflation fluid present in the bladder may cause another portion of the bladder to bulge with may cause discomfort or rupture the bladder. However, when the inflation devices are in fluid communication with each other, the inflation fluid displaced by the compression may be distributed over a larger area thereby preventing or at least minimizing the formation of bulges.

In an embodiment, as illustrated, the tube 438 may extending within the chamber 406. However, extending the tube 438 within the chamber 406 may decrease the quantity of bodily fluids that may be stored in the chamber 406 and may make disposing the porous material 410 in the chamber 406 more difficult. In an embodiment, the tube 438 may extend within the fluid impermeable barrier 402 (e.g., the fluid impermeable barrier 402 forms the tube 438) or extends outside of the chamber 406 and the fluid impermeable barrier 402. In an embodiment, the at least one tube 438 includes a plurality of tubes 438. In such an embodiment, at least one (e.g., all) of the plurality of tubes 438 may be disposed in the chamber 406, within the fluid impermeable barrier 402, outside of the chamber 406 and the fluid impermeable barrier 402, or combinations thereof.

In an embodiment, as illustrated, each of the first and second inflation devices 412a, 412b are in fluid communication with each other. In such an embodiment, the fluid collection assembly 400 may only include a single valve 420 which is used to inflate and deflate each of the first and second inflation devices 412a, 412b. It is noted that the fluid collection assembly 400 may include a plurality of valves 420 even when each of the first and second inflation devices 412a, 412b are in fluid communication with each other. In an embodiment, at least some of the plurality of inflation devices are not in fluid communication with each other. For example, when the first and second inflation devices 412a, 412b each include two or more inflation devices, the first inflation devices 412a may be in fluid communication with each other and the second inflation devices 412b may be in fluid communication with each other. As such, the length and width of the fluid collection assembly 400 may be control independently from each other.

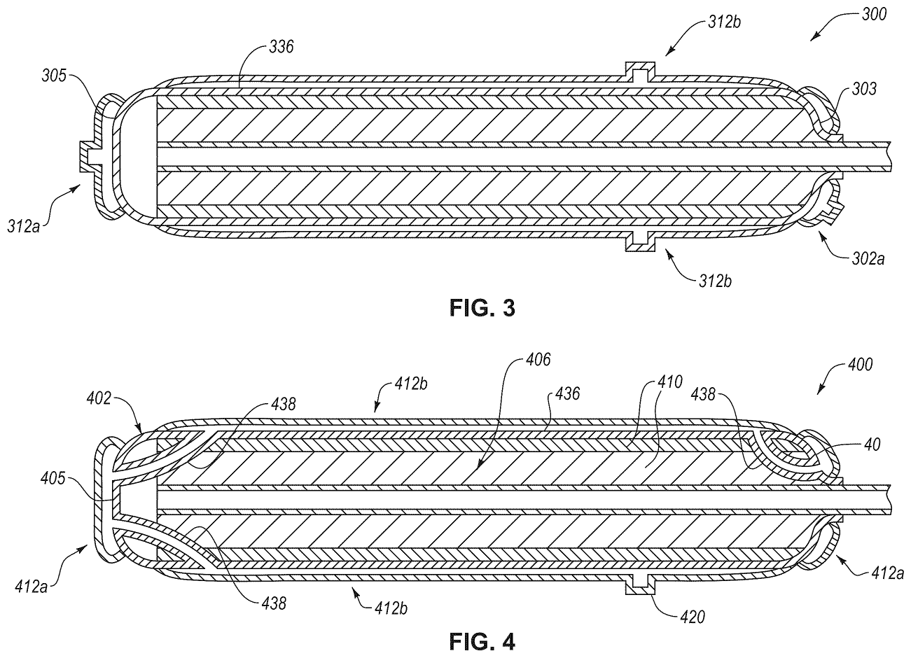

In an embodiment, the fluid collection assemblies disclosed herein may include a single inflation device that is configured to controllably change the length and width of the fluid collection assembly. FIG. 5 is a cross-sectional schematic of a fluid collection assembly 500 including a single inflation device 512 that is configured to controllably change the length and width of the fluid collection assembly 500, according to an embodiment. Except as otherwise disclosed herein, the fluid collection assembly 500 is the same as or substantially similar to any of the fluid collection assemblies disclosed herein.

The inflation device 512 may include a single bladder 514 and at least one valve 520. The bladder 514 includes one or more walls 516 defining a single interior region 518 and the valve 520 is in fluid communication with the interior region such that the valve 520 may add the inflation fluid to or remove inflation fluid from the interior region 518. The inflation device 512 is positioned adjacent to at least one of the proximal end region 503 or the distal end region 505 of the fluid impermeable barrier 502. The inflation device 512 is also positioned adjacent to at least a portion of the lateral side 536 of the fluid impermeable barrier 502 on either one or both sides of the opening (not shown). As such, the inflation device 512 is able to control the length and the width of the fluid collection assembly 500. Further, the inflation device 512 may exhibit a relatively large size compared to an inflation device that was only adjacent to one of the proximal end region 503, the distal end region 505, or a portion of the lateral side 536. The greater size of the inflation device 512 allows the inflation fluid to be distributed therein without or minimizing bulging when movement of the individual using the fluid collection assembly 500 compresses a portion of the inflation device.

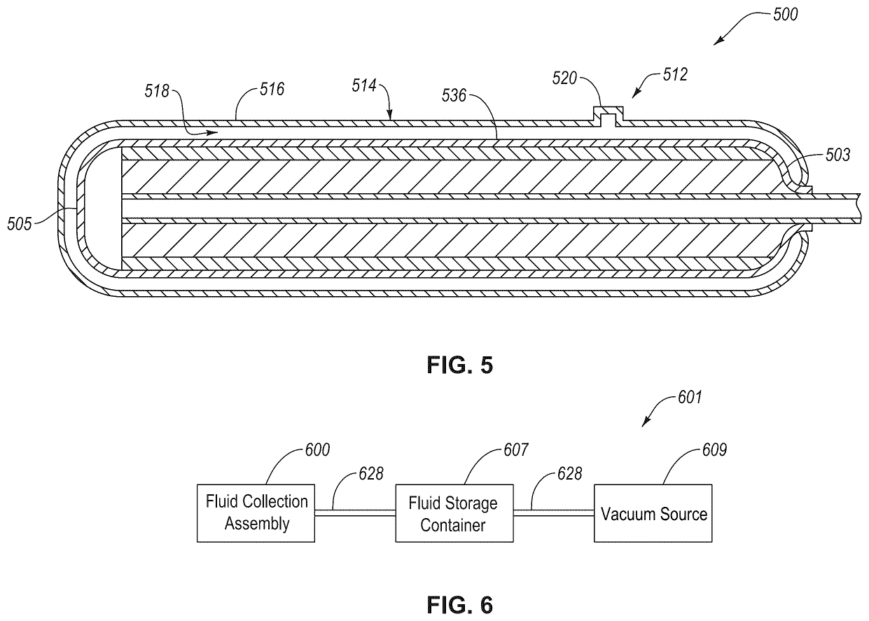

FIG. 6 is a block diagram of a system 601 for fluid collection, according to an embodiment. The system 601 includes a fluid collection assembly 600, a fluid storage container 607, and a vacuum source 609. The fluid collection assembly 600, the fluid storage container 607, and the vacuum source 609 may be fluidly coupled to each other via one or more conduits 628. For example, fluid collection assembly 600 may be operably coupled to one or more of the fluid storage container 607 or the vacuum source 609 via the conduit 628. Fluid (e.g., urine or other bodily fluids) collected in the fluid collection assembly 600 may be removed from the fluid collection assembly 600 via the conduit 628 which protrudes into the fluid collection assembly 600. For example, an inlet of the conduit 628 may extend into the fluid collection assembly 600, such as to a reservoir. The outlet of the conduit 628 may extend into the fluid collection assembly 600 or the vacuum source 609. Suction may be introduced into the chamber of the fluid collection assembly 600 via the inlet of the conduit 628 responsive to suction (e.g., vacuum) force applied at the outlet of the conduit 628.

The suction force may be applied to the outlet of the conduit 628 by the vacuum source 609 either directly or indirectly. The suction force may be applied indirectly via the fluid storage container 607. For example, the outlet of the conduit 628 may be disposed within the fluid storage container 607 and an additional conduit 628 may extend from the fluid storage container 607 to the vacuum source 609. The vacuum source 609 may apply suction to the fluid collection assembly 600 via the fluid storage container 607. The suction force may be applied directly via the vacuum source 609. For example, the outlet of the conduit 628 may be disposed within the vacuum source 609. An additional conduit 628 may extend from the vacuum source 609 to a point outside of the fluid collection assembly 600, such as to the fluid storage container 607. In such examples, the vacuum source 609 may be disposed between the fluid collection assembly 600 and the fluid storage container 607.

The fluid collection assembly 600 may be similar or identical to any of the fluid collection assemblies disclosed herein in one or more aspects. For example, the fluid collection assembly 600 may include a fluid impermeable barrier including a proximal end region, a distal end region, and at least one lateral side. The fluid impermeable barrier defines at least one opening, a chamber, and a fluid outlet at the proximal end region. The fluid collection assembly may at least one include at least one porous material and at least one inflation device. The inflation device is configured to controllably change a length and/or width of the fluid collection assembly.

The fluid storage container 607 is sized and shaped to retain a fluid. The fluid storage container 607 may include a bag (e.g., drainage bag), a bottle or cup (e.g., collection jar), or any other enclosed container for storing bodily fluid(s) such as urine. In some examples, the conduit 628 may extend from the fluid collection assembly 600 and attach to the fluid storage container 607 at a first point. An additional conduit 628 may attach to the fluid storage container 607 at a second point thereon and may extend and attach to the vacuum source 609. A vacuum (e.g., suction) may be drawn through fluid collection assembly 600 via the fluid storage container 607. Fluid, such as urine, may be drained from the fluid collection assembly 600 using the vacuum source 609.

The vacuum source 609 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 609 may provide a vacuum or suction to remove fluid from the fluid collection assembly 600. In some examples, the vacuum source 609 may be powered by one or more of a power cord (e.g., connected to a power socket), one or more batteries, or even manual power (e.g., a hand operated vacuum pump). In some examples, the vacuum source 609 may be sized and shaped to fit outside of, on, or within the fluid collection assembly 600. For example, the vacuum source 609 may include one or more miniaturized pumps or one or more micro pumps. The vacuum sources 609 disclosed herein may include one or more of a switch, a button, a plug, a remote, or any other device suitable to activate the vacuum source 609.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. (canceled)

2. A fluid collection assembly, comprising:
a fluid impermeable barrier defining a chamber, at least one opening, and at least one fluid outlet;
at least one porous material at least partially disposed in the chamber; and
at least one inflatable bladder configurable between a first state and at least a second state;
wherein the fluid impermeable barrier exhibits a first dimension measured when the at least one inflatable bladder exhibits the first state and a second dimension when the at least one inflatable bladder exhibits the second state, the first dimension less than the second dimension, the first dimension and the second dimension are a width measured perpendicular to a longitudinal axis of the fluid collection assembly or a length measured parallel to the longitudinal axis; and
wherein, when the fluid collection assembly is disposed adjacent to a urethra opening of an individual, switching the at least one inflatable bladder between the first state and at least the second state causes the fluid collection assembly to better conform to one or more anatomical features of the individual.

3. The fluid collection assembly of claim 2, wherein the at least one inflatable bladder extends generally parallel to longitudinal axis of the fluid impermeable barrier.

4. The fluid collection assembly of claim 2, wherein:
the fluid impermeable barrier includes two lateral sides on opposing sides of the at least one opening; and
at least a portion of the at least one inflatable bladder is adjacent to at least one of the two lateral sides.

5. The fluid collection assembly of claim 4, wherein the first dimension and the second dimension are the width.

6. The fluid collection assembly of claim 5, wherein switching the at least one inflatable bladder between the first state and the second state decreases gaps between the labia folds and the fluid collection assembly.

7. The fluid collection assembly of claim 4, wherein the two lateral sides and the at least one inflatable bladder exhibit an elongated shape and extend parallel to each other.

8. The fluid collection assembly of claim 4, wherein the at least one inflatable bladder contacts an outer surface of the two lateral sides of the fluid impermeable barrier.

9. The fluid collection assembly of claim 4, wherein the at least one inflatable bladder is spaced from the at least one opening.

10. The fluid collection assembly of claim 4, wherein the fluid impermeable barrier includes a proximal end region and a distal end region opposite the proximal end region, the at least one inflatable bladder is spaced from the proximal end region and the distal end region.

11. The fluid collection assembly of claim 2, wherein the first dimension and the second dimension are the length.

12. The fluid collection assembly of claim 2, wherein the at least one inflatable bladder including a first inflatable bladder and a second inflatable bladder; and
wherein the fluid impermeable barrier exhibits the first dimension and the second dimension when the first inflatable bladder or the second inflatable bladder exhibits the first state and the second state, respectively.

13. The fluid collection assembly of claim 12, wherein one of the first inflatable bladder or the second inflatable bladder includes at least one valve.

14. The fluid collection assembly of claim 12, wherein each of the first inflatable bladder and the second inflatable bladder includes at least one valve.

15. The fluid collection assembly of claim 12, wherein the first inflatable bladder is in fluid communication with the second inflatable bladder.

16. The fluid collection assembly of claim 2, wherein the first dimension is less than the second dimension by about 1 cm or less.

17. The fluid collection assembly of claim 2, wherein the first dimension is less than the second dimension by about 5 mm or less.

18. The fluid collection assembly of claim 2, wherein the at least one inflatable bladder is spaced from the chamber.

19. The fluid collection assembly of claim 2, wherein the at least one inflatable bladder is closer to the at least one opening than a back side of the fluid impermeable barrier, the back side of the fluid impermeable barrier is opposite the at least one opening.

20. The fluid collection assembly of claim 2, wherein the at least one inflatable bladder is configured to substantially only contact the labia folds.

21. The fluid collection assembly of claim 2, further comprising a conduit in fluid communication with the chamber, wherein the conduit is distinct and separate from the at least one inflatable bladder.

22. The fluid collection assembly of claim 2, further comprising a conduit configured to have one or more bodily fluids flow therein, the inflatable bladder including at least one valve and the at least one valve is spaced from and not in fluid communication with the conduit.

23. The fluid collection assembly of claim 2, further comprising at least one additional inflatable bladder contacting at least an outer surface of a distal end region of the fluid impermeable barrier, the at least one inflatable bladder configured to change a length of the fluid impermeable barrier responsive to flowing at least one inflation fluid into the bladder of the at least one additional inflation device.

24. A system, comprising:
a fluid collection assembly including:
a fluid impermeable barrier defining a chamber, at least one opening, and at least one fluid outlet;
at least one porous material at least partially disposed in the chamber; and
at least one inflatable bladder configurable between a first state and at least a second state;
wherein the fluid impermeable barrier exhibits a first dimension measured when the at least one inflatable bladder exhibits the first state and a second dimension when the at least one inflatable bladder exhibits the second state, the first dimension less than the second dimension, the first dimension and the second dimension are a width measured perpendicular to a longitudinal axis of the fluid collection assembly or a length measured parallel to the longitudinal axis; and
wherein, when the fluid collection assembly is disposed adjacent to a urethral opening of an individual, switching the at least one inflatable bladder between the first state and at least the second state causes the fluid collection assembly to better conform to one or more anatomical features of the individual;
a fluid storage container; and
a vacuum source;
wherein the at least one fluid outlet of the fluid collection assembly, the fluid storage container, and the vacuum source are in fluid communication with each other.

25. A method of using a fluid collection assembly, the method comprising:
positioning at least one opening of the fluid collection assembly adjacent to a urethral opening, the fluid collection assembly including:
a fluid impermeable barrier defining a chamber, at least one opening, and at least one fluid outlet;
at least one porous material at least partially disposed in the chamber; and
at least one inflatable bladder configurable between a first state and at least a second state;
wherein the fluid impermeable barrier exhibits a first dimension measured when the at least one inflatable bladder exhibits the first state and a second dimension when the at least one inflatable bladder exhibits the second state, the first dimension less than the second dimension, the first dimension and the second dimension are a width measured perpendicular to a longitudinal axis of the fluid collection assembly or a length measured parallel to the longitudinal axis; and
switching the at least one inflatable bladder from the first state to the second state or from the second state to the first state to cause the fluid collection assembly to better conform to one or more anatomical features.
